(19) United States
(12) Patent Application Publication
(10) Pub. No.: US 2024/0301510 A1
(43) Pub. Date: Sep. 12, 2024

(54) ASSESSING RISK FOR COLORECTAL ADENOMA RECURRENCE BY NONINVASIVE MEANS

(71) Applicant: The Chinese University of Hong Kong, Shatin, New Territories (CN)

(72) Inventors: Siew Chien NG, Shatin New Territories (CN); Ka Leung Francis CHAN, Tai Po, New (CN); Qiaoyi LIANG, Tuen Mun, New Territories (CN)

(21) Appl. No.: 18/037,693

(22) PCT Filed: Nov. 18, 2021
(86) PCT No.: PCT/CN2021/131499

§ 371 Date: May 18, 2023

Related U.S. Application Data
(60) Provisional application No. 63/196,582, filed on Jun. 3, 2021.
(60) Provisional application No. 63/116,104, filed on Nov. 19, 2020.

(51) Int. Cl. C12Q 1/689; C12N 15/113; C12Q 1/6886

(52) U.S. Cl. CPC C12Q 1/689; C12N 15/113; C12Q 1/6886; C12N 2320/30; C12Q 2600/118

(57) ABSTRACT

A noninvasive methods for assessing the likelihood of recurrence of colorectal adenoma among patients who have previously undergone a procedure for colorectal cancer or adenoma removal as well as methods for reducing risk of colorectal adenoma recurrence in these patients. Kits useful for such methods are also provided.

### RELATED APPLICATIONS

This application claims priority to U.S. Provisional Patent Application No. 63/116,104, filed Nov. 19, 2020, and U.S. Provisional Patent Application No. 63/196,582, filed Jun. 3, 2021, the contents of each of the above are hereby incorporated by reference in the entirety for all purposes.

### BACKGROUND OF THE INVENTION

Colorectal cancer (CRC) is the third most common cancer and the third leading cause of cancer mortality worldwide. Both the incidence and death rate of CRC are increasing rapidly and maintaining an upward trend in Asian countries, despite ongoing efforts devoted towards the control of new CRC incidence. The global persistence of CRC necessitates a paradigm shift in its management strategy, from clinical treatment to preclinical prevention. It is of critical importance that colorectal adenoma, precancerous growth in the form of polyps or cysts, is detected early and properly treated in order to prevent the progression to colorectal cancer. Thus, there exists an urgent need for new and reliable methods to assess among patients, especially those who have previously undergone polypectomy, the likelihood of recurrence of colorectal adenoma.

Microbial markers were previously shown to be useful for noninvasive diagnosis of colorectal cancer and adenoma. It was unclear, however, whether such biomarkers can serve to predict adenoma recurrence in patients after removal of advanced adenoma. The present inventors studied microbial markers, Fusobacterium nucleatum (Fn), Bacteroides clarus (Bc), Clostridium hathewayi (Ch), Roseburia intestinalis (Ri), and the Lachnoclostridium marker m3, in the stool from patients before and after colonoscopy where colorectal adenoma was removed. Significant increases in m3, Fn, and Ch, and the combined score of 4Bac (Fn, m3, Bc and Ch) panel were detected as positively associated with recurrence. Thus, these fecal microbial markers can be used in noninvasive prediction of the risk of colorectal adenoma recurrence as well as diagnosis of adenoma recurrence. As such, this discovery provides a new, important, and improved means for the early detection colorectal adenoma and effective prevention of colorectal cancer while requiring no invasive measure.

### BRIEF SUMMARY OF THE INVENTION

The present inventors have discovered a correlation between the recurrence of colorectal adenoma in a patient who has previously had polypectomy and elevated level of certain bacterial species. Thus, the first aspect of the present invention provides a method for assessing the risk of a patient experiencing recurring colorectal adenoma after a prior procedure to remove colorectal cancer or adenoma. The method includes these steps: (a) obtaining in a first stool sample taken from an individual prior to the removal of colorectal cancer or adenoma a baseline level of one or more of the bacterial species Fusobacterium nucleatum (Fn), a Lachnoclostridium species carrying genetic marker m3 (m3), Bacteroides clarus (Bc), and Clostridium hathewayi (Ch); (b) obtaining in a second stool sample taken from the individual after the removal of colorectal cancer or adenoma a follow-up level of one or more of Fn, m3, Bc, and Ch; (c) calculating a combined score from the baseline level and follow-up level of any one of more of the four bacterial species Fn, m3, Bc, and Ch by a method specified in Table 1; and (d) detecting the value from step (c) to be higher than a standard control value and determining the individual as having increased risk for colorectal adenoma recurrence. On the other hand, when the follow-up value is no higher than a standard control value, the individual is deemed as having no increased risk for colorectal adenoma recurrence. The standard control value is the combined score calculated from the baseline levels and follow-up levels of one (Fn or m3 or Ch), two (m3 and Fn, or m3 and Ch), three (Fn, m3, and Ch), or four bacterial species Fn, m3, Bc, and Ch) that is established from a control group of individuals who have experienced no colorectal adenoma recurrence, e.g., for about 3 to 10 years, after removal of colorectal cancer or adenoma. In some cases, the claimed method for assessing the risk of a patient experiencing recurring colorectal adenoma after a prior procedure to remove colorectal cancer or adenoma is performed according to the above-described steps by measuring the level of one or more bacterial markers determined based on the level of one or more of the nucleotide sequence of SEQ ID NO: 19, SEQ ID NO:20, SEQ ID NO:21, and SEQ ID NO:22.

Similarly, a further method is provided for assessing risk for colorectal adenoma recurrence in an individual after removal of colorectal cancer or adenoma. The method comprises the steps of: (a) obtaining in a stool sample taken from the individual after the removal of colorectal cancer or adenoma a value of (1) level of one or more of three bacterial species of a Lachnoclostridium species carrying genetic marker m3 (m3), Fusobacterium nucleatum (Fn), and Clostridium hathewayi (Ch); or (2) combined score of levels of two bacterial species m3 and Ch, which is calculated by I₂+β₅*m3+β₆*Ch; or (3) combined score of levels of three bacterial species Fn, m3, and Ch, which is calculated by I₃+β₇*Fn+β₈*m3+β₉*Ch; or (4) combined score of levels of four bacterial species Fn, m3, Bc, and Ch, which is calculated by I₁+β₁*Fn+β₂*m3+β₃*Bc+β₄*Ch; and (b) detecting the value from step (a) to be higher than a standard control value and determining the individual as having increased risk for colorectal adenoma recurrence. The standard control value is a value of the same category (i.e., either the level of Fn) or m3 or Ch; or the combined score calculated from the levels of two (m3 and Ch), three (Fn, m3, and Ch), or four bacterial species Fn, m3, Bc, and Ch) that is established from a control group of individuals who have experienced no colorectal adenoma recurrence, e.g., for about 3 to 10 years, after removal of colorectal cancer or adenoma. In some cases, the claimed method for assessing the risk of a patient experiencing recurring colorectal adenoma after a prior procedure to remove colorectal cancer or adenoma is performed according to the above-described steps by measuring the level of one or more bacterial markers determined based on the level of one or more of the nucleotide sequence of SEQ ID NO:19, SEQ ID NO:20, SEQ ID NO:21, and SEQ ID NO:22.

In some embodiments of either of the two methods described above, the individual had colorectal adenoma such as polyps or cysts removed by polypectomy, or had CRC removed by surgery. In some embodiments, the combined score of the levels of any two, three, or four of the four bacterial species Fn, m3, Bc, and Ch is calculated by a method specified in Table 1. In some embodiments, the genome of m3 comprises the nucleotide sequence of SEQ ID NO: 19. In some embodiments, the genome of Ch comprises the nucleotide sequence of SEQ ID NO:20. In some embodiments, the genome of Fn comprises the nucleotide sequence of SEQ ID NO:21. In some embodiments, the genome of Bc comprises the nucleotide sequence of SEQ ID NO:22. In some embodiments of either method, each of step (a) and/or (b) comprises obtaining the level of a DNA, RNA, or protein unique to at least one of the bacterial species Fn, m3, Bc, and Ch. In some embodiments of either method, each of step (a) and/or (b) comprises a polymerase chain reaction (PCR), such as a quantitative polymerase chain reaction (qPCR) or reverse transcription polymerase chain reaction (RT-PCR), for measuring the level or levels of the bacterial species. In some embodiments of either method, the post-removal stool sample is taken from the individual about one to about five years after the initial removal of the colorectal cancer or adenoma, for example, the stool sample is taken from the individual about one year after the removal of the colorectal cancer or adenoma. In some embodiments of either method, the method further comprises, upon determining the individual as having increased risk for colorectal adenoma recurrence in step (b) or (c), a step of performing regular (e.g., yearly) colonoscopy to monitor the individual or administering to the individual an inhibitor that suppresses or eliminates one or more of the bacterial species Fn, m3, Bc, and Ch in the individual, especially in the gastrointestinal tract. In some embodiments of either method, the inhibitor is a small inhibitory RNA or antisense oligonucleotide specifically targeting at least one gene of the one or more of the bacteria species Fn, m3, Bc, and Ch, or an expression cassette directing expression of the inhibitory RNA, or a viral vector comprising the expression cassette.

In a second aspect, the present invention provides a kit for assessing risk for colorectal adenoma recurrence in an individual after removal of colorectal cancer or adenoma, comprising (1) a first container containing a reagent for measuring level of the bacterial species Fn; and (2) a second container containing a reagent for measuring level of the bacterial species m3. In some embodiments, the kit further comprises a third container containing one or more reagents for measuring level of the bacterial species Bc. In some embodiments, the kit further comprises a third container containing one or more reagents for measuring level of the bacterial species Ch.

In some embodiments, the reagent in each of the containers is a reagent for a polymerase chain reaction (PCR), for example, a qPCR or RT-PCR. In some embodiments, the reagent in each of the containers is a reagent for detecting a protein unique to the bacterial species such as Fn, m3, Bc, or Ch.

In a third aspect, the present invention provides a method for reducing risk of colorectal adenoma recurrence in an individual after removal of colorectal cancer or adenoma. The method comprises administering to the individual an effective amount of an inhibitor that suppresses or eliminates one or more of the bacterial species Fn, m3, and Ch in the individual, especially in the gastrointestinal tract.

In some embodiments, the inhibitor is a small inhibitory RNA or antisense oligonucleotide specifically targeting at least one gene of the one or more of the bacterial species Fn, m3, and Ch, or an expression cassette directing expression of the inhibitory RNA, or a viral vector comprising the expression cassette. In some embodiments, the expression cassette is comprised within a viral particle. In some embodiments, the method further comprises, after the administering step, a step of determining level of the one or more of the bacterial species Fn, m3, and Ch in the individual's stool. In some embodiments, the administering step is taken within about one year after the removal of the colorectal cancer or adenoma. In some embodiments, the administering step is taken multiple times (e.g., once every year) within a period of from about one year to about five or ten years after the initial removal of the colorectal cancer or adenoma. In some embodiments, the targeted gene is within the nucleotide sequence of SEQ ID NO: 19 (e.g., in the m3 genome), or within the nucleotide sequence of SEQ ID NO:20 (e.g., in the Ch genome), or within the nucleotide sequence of SEQ ID NO:21 (e.g., in the Fn genome).

In a fourth aspect, the present invention provides a kit for reducing the risk of colorectal adenoma recurrence, comprising (1) a first container containing one or more reagents for measuring level of the one or more of the bacterial species Fn, m3, and Ch; and (2) a second container containing a composition comprising an effective amount of an inhibitor that suppresses or eliminates one or more of the bacterial species Fn, m3, and Ch. In some embodiments, the first container comprises PCR reagents for measuring the level of a DNA or RNA of the one or more of the bacterial species Fn, m3, and Ch, such as primers or probes for PCR, e.g., qPCR or RT-PCR. In some embodiments, the inhibitor is a small inhibitory RNA or antisense oligonucleotide specifically targeting at least one gene of the one or more of the bacterial species Fn, m3, and Ch, or an expression cassette directing expression of the inhibitory RNA, or a viral vector comprising the expression cassette.

### BRIEF DESCRIPTION OF THE DRAWINGS

FIG. 1. Changes in fecal bacterial markers in post-polypectomy subjects as compared to patients with baseline advanced adenoma. (FIG. 1A) Subject recruitment strategy and categories of samples/subjects in this study. (FIG. 1B) Comparison of the levels of four fecal bacterial markers between baseline stool samples collected before index colonoscopic diagnosis of advanced adenoma and follow-up stools before surveillance colonoscopy from subjects who developed recurrent adenoma (R) and those without recurrence (no-R) in group III subjects with paired baseline and follow-up stools. (FIG. 1C) Comparison of the levels of four fecal bacterial markers between baseline stool samples and follow-up samples from all subjects recruited in this study. (FIG. 1D) In fecal samples of patients with baseline adenoma (D1) and those with recurrent adenoma (D2), levels of m3, Fn and Ch showed no difference between patients with proximal lesions and those with distal lesions. Fn, Fusobacterium nucleatum; m3, Lachnoclostridium marker m3; Ch, Clostridium hathewayi; Bc, Bacteroides clarus.

FIG. 2. Comparison of the baseline (FIG. 2A) and follow-up (FU) (FIG. 2B) levels of four bacterial markers and their combined score 4Bac between patients with and without recurrent adenoma. Only patients with both baseline and FU stools were included here to assess the effects of baseline and FU marker levels in predicting recurrent adenoma. no-R, no recurrence; R, recurrence; Fn, Fusobacterium nucleatum; m3, Lachnoclostridium marker m3; Ch, Clostridium hathewayi; Bc, Bacteroides clarus; 4Bac: combined score of Fn, m3, Ch and Bc by logistic regression.

FIG. 3. Diagnostic performances of bacterial markers during follow-up (FU) surveillance in predicting recurrent adenoma. (FIG. 3A) Receiver operating characteristic (ROC) curve analyses and diagnostic performances of Fusobacterium nucleatum (Fn), the Lachnoclostridium marker m3, Clostridium hathewayi (Ch) and the combined score 4Bac in distinguishing patients with and without recurrent adenoma. CI, confidence interval; PPV, positive predictive value; NPV, negative predictive value. (FIG. 3B) Performances of logistic regression models involving different marker panels in discriminating patients with recurrence from those without recurrence. AUROC, area under ROC; no-R, no recurrence; R, recurrence.

FIG. 4. Changes in bacterial markers at follow-up (FU) versus baseline predict recurrence of adenoma. (FIG. 4A) The four bacterial markers and their combined score 4Bac showed no significant difference between baseline and FU stools in patients without recurrence. Significant increases in m3 and 4Bac were detected in FU stools as compared to baseline stools in patients with recurrence. (FIG. 4B) Performances of logistic regression models involving changes in FU stools versus baseline stools in discriminating patients with recurrence from those without recurrence. The model combining changes in Fn, m3 and Ch showed the best performance. Fn, Fusobacterium nucleatum; m3, Lachnoclostridium marker m3; Ch, Clostridium hathewayi; Bc, Bacteroides clarus; 4Bac: combined score of Fn, m3, Ch and Bc previously devised for diagnosis of CRC and adenoma. no-R, no recurrence; R, recurrence.

FIG. 5. Validation of the new logistic regression model involving levels of Fusobacterium nucleatum (Fn), the Lachnoclostridium marker m3, Clostridium hathewayi (Ch) in follow-up stools for diagnosis of recurrent adenoma. FIG. 5(A) Diagnostic performances of the new model (A1) and FIT (A2) as referring to results determined by colonoscopy and histological examinations. FIG. 5(B) Comparison of the combined score between patients with recurrent adenoma and subjects without recurrence and ROC curve analysis for the combined score. R, with recurrence; no-R, without recurrence.

### DEFINITIONS

In this disclosure the terms “colorectal cancer (CRC)” and “colon cancer” have the same meaning and refer to a cancer of the large intestine (colon), the lower part of human digestive system, although rectal cancer often more specifically refers to a cancer of the last several inches of the colon, the rectum. A “colorectal cancer cell” is a colon epithelial cell possessing characteristics of colon cancer and encompasses a precancerous cell, which is in the early stages of conversion to a cancer cell or which is predisposed for conversion to a cancer cell. Such cells may exhibit one or more phenotypic traits characteristic of the cancerous cells.

As used herein, the term “colorectal adenoma” refers to a pre-cancerous growth or precursor of CRC in the form of a polyp or cyst that can progress to CRC if left untreated, typically by removal via colonoscopy, such as polypectomy, or by a surgical procedure.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins of interest, wherein the amino acid residues are linked by covalent peptide bonds.

The term “amino acid” refers to refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in WO01/12654, which may improve the stability (e.g., half-life), bioavailability, and other characteristics of a polypeptide comprising one or more of such D-amino acids. In some cases, one or more, and potentially all of the amino acids of a therapeutic polypeptide have D-chirality.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

As used herein, the term “gene expression” is used to refer to the transcription of a DNA to form an RNA molecule encoding a particular protein or the translation of a protein encoded by a polynucleotide sequence. In other words, both mRNA level and protein level encoded by a gene of interest are encompassed by the term “gene expression level” in this disclosure.

An “expression cassette” is a nucleic acid construct, generated recombinantly or synthetically, with a series of specified nucleic acid elements that permit transcription of a particular polynucleotide sequence in a host cell, for example, the transcription of an inhibitory RNA (e.g., such as miRNA or siRNA) or an anti-sense RNA targeting a specific, pre-selected sequence (e.g., a segment of Fusobacterium nucleatum (Fn), Bacteroides clarus (Bc), Clostridium hathewayi (Ch), or a Lachnoclostridium species bearing marker m3 (m3) genomic sequence). An expression cassette may be part of a plasmid, viral genome, or nucleic acid fragment. In other words, an expression cassette may be transferred or delivered as a part or/in the form of a bacterial plasmid or a viral vector or virus-like particle. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used in this application, an “increase” or a “decrease” refers to a detectable positive or negative change in quantity from a comparison control, e.g., an established standard control or cut-off value or baseline value. An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” “lower,” “larger,” and “smaller,” are used in this application in the same fashion as described above. In contrast, the term “substantially the same” or “substantially lack of change” indicates little to no change in quantity from the standard control value, typically within ±10% of the standard control, or within ±5%, 2%, 1%, or even less variation from the standard control.

The term “inhibiting” or “inhibition,” as used herein, refers to any detectable negative effect on a target biological process, such as RNA transcription, protein expression, cell proliferation, cellular signal transduction, cell proliferation, tumorigenicity, metastatic potential, and recurrence of a disease/condition. Typically, an inhibition is reflected in a decrease of at least 10%, 20%, 30%, 40%, or 50% in target process (e.g., level of a pertinent bacterium such as Fn, Bc, Ch, or m3; or the incidence of colorectal adenoma occurrence) upon application of an inhibitor, when compared to a control value where the inhibitor is not applied.

“Primers” as used herein refer to oligonucleotides that can be used in an amplification method, such as a polymerase chain reaction (PCR), to amplify a nucleotide sequence based on the polynucleotide sequence corresponding to a gene of interest, e.g., a unique polynucleotide sequence from a pertinent bacterial species such as gene markers m1704941 (from Fn), m370640 (from Bc), and m2736705 (from Ch), see, e.g., WO2018/036503. For example, “primers” can be used in a reverse-transcription polymerase chain reaction (RT-PCR) and quantitative polymerase chain reaction (qPCR) to quantify the gene expression. Typically, at least one of the PCR primers for amplification of a polynucleotide sequence is sequence-specific for that polynucleotide sequence. The exact length of the primer will depend upon many factors, including temperature, source of the primer, and the method used. For example, for diagnostic and prognostic applications, depending on the complexity of the target sequence, the oligonucleotide primer typically contains at least 10, or 15, or 20, or 25 or more nucleotides, although it may contain fewer nucleotides or more nucleotides. The factors involved in determining the appropriate length of primer are readily known to one of ordinary skill in the art. The primers used in particular embodiments are shown in Examples of the disclosure where their specific applications are indicated. In this disclosure the term “primer pair” means a pair of primers that hybridize to opposite strands a target DNA molecule or to regions of the target DNA which flank a nucleotide sequence to be amplified. In this disclosure the term “primer site”, means the area of the target DNA or other nucleic acid to which a primer hybridizes.

The term “amount” or “level” as used in this application refers to the quantity of a bacterial species of interest, e.g., Fn, Bc, Ch, or m3, present in a sample. Such quantity may be expressed in the absolute terms, i.e., the total quantity of Fn, Bc, Ch, or m3 in the sample, or in the relative terms, i.e., the percentage of Fn, Bc, Ch, or m3 among all bacteria in the sample.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of an inhibitor of a specific bacterial species such as Fn, Bc, Ch, or m3 is the amount of the inhibitor to achieve a decreased level (including to an undetectable level) of the bacterial species in a recipient's gastrointestinal tract, e.g., as measured in a stool sample obtained from the recipient. As another example, an effective amount of an inhibitor is the amount that, when administered to a patient, is able to achieve a detectable level of reduced risk of recurring colorectal adenoma in the patient. An amount adequate to achieve an intended effect in the therapeutic context is defined as the “therapeutically effective dose.” The dosing range varies with the nature of the therapeutic agent being administered and other factors such as the route of administration and the severity of a patient's condition.

The term “anti-bacterial agent” refers to any substance that is capable of inhibiting, suppressing, eliminating, or preventing the growth or proliferation of a bacterial species, respectively, such as Fn, Bc, Ch, or m3. Known agents with anti-bacterial activity include various antibiotics that generally suppress the proliferation of a broad spectrum of bacterial species as well as agents such as antisense oligonucleotides, small inhibitory RNAs, and the like that can inhibit the proliferation of specific bacterial species.

“Percentage relative abundance,” when used in the context of describing the presence of a particular bacterial species (e.g., Fn, Bc, Ch, or m3) in relation to all bacterial species, respectively, present in the same environment, refers to the relative amount of the bacterial species out of the amount of all bacterial species, respectively, as expressed in a percentage form. For instance, the percentage relative abundance of one particular bacterial species can be determined by comparing the quantity of DNA or RNA specific for this species (e.g., determined by quantitative polymerase chain reaction (PCR) including reverse transcription (RT)-PCR) in one given sample with the quantity of all bacterial DNA (e.g., determined by quantitative PCR including RT-PCR and sequencing based on 16S rRNA sequence) in the same sample.

“Absolute abundance,” when used in the context of describing the presence of a particular bacterial species (e.g., Fn, Bc, Ch, or m3) in a sample (e.g., a stool sample taken from a test subject), refers to the amount of DNA derived from the bacterial species, respectively, out of the amount of all DNA in the sample. For instance, the absolute abundance of one bacterium can be determined by comparing the quantity of DNA specific for this bacterial species (e.g., determined by quantitative PCR including RT-PCR) in one given sample with the quantity of all DNA in the same sample.

“Total bacterial load” of a sample, as used herein, refers to the amount of all bacterial DNA, respectively, out of the amount of all DNA in the sample. For instance, the absolute abundance of bacteria can be determined by comparing the quantity of bacterial specific DNA (e.g., 16S rRNA determined by quantitative RT-PCR) in one given sample with the quantity of all DNA in the same sample.

“Inhibitors,” “activators,” and “modulators” of a pertinent bacterial species (such as Fn, Bc, Ch, or m3) refer to inhibitory, activating, or modulating molecules, respectively, identified using in vitro and in vivo assays for their capability to positively or negatively modulate the bacterium's proliferation or survival. The term “modulator” includes inhibitors and activators. Inhibitors are agents that, e.g., partially or totally block binding, decrease, prevent, delay activation, inactivate, desensitize, or down regulate the level or amount of the pertinent protein, potentially by suppressing downstream effects such as the growth or survival of the colorectal cancer cells. In some cases, the inhibitor directly or indirectly binds to a target DNA or RNA, such as an antisense molecule or micro RNA. Inhibitors, as used herein, are synonymous with inactivators and antagonists. Activators are agents that, e.g., stimulate, increase, facilitate, enhance activation, sensitize or up regulate the level or amount of a pertinent protein, potentially by promoting downstream effects such as the growth or survival of the colorectal cancer cells. Inhibitors, activators, and modulators can be macromolecules such as polynucleotides, polypeptides including antibodies and antibody fragments, or they can be small molecules including carbohydrate-containing molecules, siRNAs, RNA aptamers, and the like.

“Standard control” as used herein refers to a value corresponding to either the average level of a pre-selected bacterial species found in a particular type of samples (e.g., stool samples) obtained from individuals who did not suffer from colorectal adenoma recurrence or a composite score calculated from the average levels of multiple bacterial species found in the type of samples taken from such individuals. For example, for the purpose of examining a patient who has earlier undergone a colorectal cancer or adenoma removal procedure, a “standard control” value is established to provide a cut-off value to indicate whether or not the patient being examined has an elevated risk for colorectal adenoma recurrence. In order for a “standard control” to be properly established, a sufficient number of individuals (e.g., at least 10, 12, 15, 20, 24 or more individuals) without recurrence must be included in the control group to provide samples for determination of the average level(s) of one or more pre-selected bacterial species or the composite score calculated from the levels of multiple bacterial species representative of the risk for colorectal adenoma recurrence.

As used herein, the term “about” denotes a range of value encompassing +/−10% of a pre-determined value. For instance, “about 10” means 9 to 11.

In this disclosure the term “or” is generally employed in its sense including “and/or” unless the content clearly dictates otherwise.

### DETAILED DESCRIPTION OF THE INVENTION

### I. Introduction

Colorectal cancer (CRC) is one of the most common malignancies worldwide. It is common practice for post polypectomy patients to have follow-up colonoscopic surveillance examinations to detect adenorna recurrence at recommended intervals. On the other hand, the link between gut microbiota composition and adenoma recurrence after polypectomy has not been studied. A novel bacterial marker m3 was previously reported for noninvasive diagnosis of colorectal adenoma and cancer. In this disclosure, the present inventors identify novel fecal microbiome markers to predict risk of recurrence of colorectal adenomas after polypectomy.

This study included 104 patients from a polyp surveillance study followed from 2009 to 2019. Fecal samples were collected before index colonoscopy (baseline) and surveillance colonoscopy (>6 months after polypectomy). Recurrence was defined as new adenomas detected during colonoscopy after polypectomy. Four candidate markers including Fusobacterium nucleatum (Fn), Lachnoclostridium marker (m3), Clostridium hathewayi (Ch), and Bacteroides clarus were quantified in fecal samples at baseline and follow-up by quantitative Polymerase Chain Reaction (qPCR). Fecal immunochemical test (FIT) was performed on follow-up stools.

Each of the bacterial markers Fn, m3, and Ch detected at follow-up stool samples significantly differed between subjects who had adenoma recurrence compared with those with no recurrence (n=104; all P<0.05). Logistic regression model combining Fn, m3, and Ch showed an area under the receiver operating characteristic curve (AUROC) of 0.741 (P<0.0001) [sensitivity=81.3%; specificity=55.4%] in predicting adenoma recurrence. Adding FIT to bacterial markers did not improve diagnostic accuracy. In subjects with paired baseline and follow-up stool samples (n=43), m3 (P=0.006) and Fn (P=0.07) levels were higher in subjects who developed recurrent adenomas. Logistic regression model combining changes in fecal Fn, m3, and Ch levels at follow-up compared with baseline samples showed an AUROC of 0.950 (P<0.0001) in predicting risk of adenoma recurrence [sensitivity=90.0%; specificity=87.0%]. Thus, this study identifies a panel of novel fecal microbiome markers for prediction of adenoma recurrence after polypectomy. These noninvasive markers can potentially improve colonoscopy surveillance program.

### II. General Methodology

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

The sequence of interest used in this invention, e.g., the polynucleotide sequence of a DNA or RNA unique to a bacterial species of interest, and synthetic oligonucleotides (e.g., primers) can be verified using methods well-known in the pertinent research field, for example, the chain termination method for double-stranded templates of Wallace et al., Gene 16: 21-26 (1981).

### III. Acquisition of Samples and Analysis of Bacterial DNA or RNA

The present invention relates to measuring the level or amount of a signature DNA or RNA for one or more bacterial species found in a person's stool sample as a means to assess the risk of colorectal adenoma recurrence. Thus, the first steps of practicing this invention are to obtain a stool sample from a test subject and extract DNA or RNA from the sample.

### A. Acquisition and Preparation of Stool Samples

A stool sample is obtained from a person to be tested or monitored for recurring colorectal adenoma using a method of the present invention. Collection of a stool sample from an individual can be easily achieved either in a clinic or at patient's home. An appropriate amount of stool is collected and may be stored according to standard procedures prior to further preparation. The analysis of bacterial DNA or RNA found in a patient's stool sample according to the present invention may be performed using established techniques. The methods for preparing stool samples for nucleic acid extraction are well known among those of skill in the art. See, e.g., Yu et al., Gut. 2015 Sep. 25. pii: gutjnl-2015-309800. doi: 10.1136/gutjnl-2015-309800.

### B. Extraction and Quantitation of DNA and RNA

Methods for extracting DNA from a biological sample are well-known and routinely practiced in the art of molecular biology (e.g., described by Sambrook and Russell, Molecular Cloning: A Laboratory Manual 3d ed., 2001). RNA contamination should be eliminated to avoid interference with DNA analysis.

Likewise, there are numerous methods for extracting mRNA from a biological sample. The general methods of mRNA preparation can be followed, see, e.g., Sambrook and Russell, supra; various commercially available reagents or kits, such as Trizol reagent (Invitrogen, Carlsbad, CA), Oligotex Direct mRNA Kits (Qiagen, Valencia, CA), RNeasy Mini Kits (Qiagen, Hilden, Germany), and PolyATtract® Series 9600™ (Promega, Madison, WI), may also be used to obtain mRNA from a biological sample from a test subject. Combinations of more than one of these methods may also be used. It is essential that all contaminating DNA be eliminated from the RNA preparations. Thus, careful handling of the samples, thorough treatment with DNase, and proper negative controls in the amplification and quantification steps should be used.

### 1. PCR-Based Quantitative Determination of DNA or RNA Level

Once DNA or mRNA is extracted from a sample, the amount of a predetermined bacterial DNA or RNA (such as 16s rDNA or RNA encoded by a bacterial gene unique to the bacterial species) may be quantified. The preferred method for determining the DNA or RNA level is an amplification-based method, e.g., by polymerase chain reaction (PCR), including reverse transcription-polymerase chain reaction (RT-PCR) for RNA quantitative analysis.

While a bacterial DNA is directly subject to amplification, bacterial RNA must be first reverse transcribed. Prior to the amplification step, a DNA copy (cDNA) of the target RNA must be synthesized. This is achieved by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, M N, 1993; Egger et al., J. Clin. Microbiol. 33:1442-1447, 1995; and U.S. Pat. No. 5,075,212.

The general methods of PCR are well-known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled through a denaturing region, a primer annealing region, and an extension reaction region automatically. Machines specifically adapted for this purpose are commercially available.

Although PCR amplification of the target bacterial DNA or RNA is typically used in practicing the present invention, one of skill in the art will recognize, however, that amplification of these DNA or RNA species in a sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to quantitatively determining the amount of DNA or mRNA in the sample. For a review of branched-DNA signal amplification for direct quantitation of nucleic acid sequences in clinical samples, see Nolte, Adv. Clin. Chem. 33:201-235, 1998.

### 2. Other Quantitative Methods

The target bacterial DNA or RNA can also be detected using other standard techniques, well known to those of skill in the art. Although the detection step is typically preceded by an amplification step, amplification is not required in the methods of the invention. For instance, the DNA or RNA may be identified by size fractionation (e.g., gel electrophoresis), whether or not proceeded by an amplification step. After running a sample in an agarose or polyacrylamide gel and labeling with ethidium bromide according to well-known techniques (see, e.g., Sambrook and Russell, supra), the presence of a band of the same size as the standard comparison is an indication of the presence of a target DNA or RNA, the amount of which may then be compared to the control based on the intensity of the band. Alternatively, oligonucleotide probes specific to the target bacterial DNA or RNA can be used to detect the presence of such DNA or RNA species and indicate the amount of bacterial DNA or RNA in comparison to the standard comparison, based on the intensity of signal imparted by the probe.

Sequence-specific probe hybridization is a well-known method of detecting a particular nucleic acid comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch.

A number of hybridization formats well known in the art, including but not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

The hybridization complexes are detected according to well-known techniques. Nucleic acid probes capable of specifically hybridizing to a target nucleic acid, i.e., a bacterial 16s rDNA, can be labeled by any one of several methods typically used to detect the presence of hybridized nucleic acids. One common method of detection is the use of autoradiography using probes labeled with ³H, ¹²⁵I, ³⁵, ¹⁴C, or ³²P, or the like. The choice of radioactive isotope depends on research preferences due to ease of synthesis, stability, and half-lives of the selected isotopes. Other labels include compounds (e.g., biotin and digoxigenin), which bind to antiligands or antibodies labeled with fluorophores, chemiluminescent agents, and enzymes. Alternatively, probes can be conjugated directly with labels such as fluorophores, chemiluminescent agents or enzymes. The choice of label depends on sensitivity required, ease of conjugation with the probe, stability requirements, and available instrumentation.

The probes and primers necessary for practicing the present invention can be synthesized and labeled using well-known techniques. Oligonucleotides used as probes and primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides is by either native acrylamide gel electrophoresis or by anion-exchange HPLC as described in Pearson and Regnier, J. Chrom., 255:137-149, 1983.

### IV. Quantitation of Bacterial Proteins

### A. Preparing Samples for Bacterial Protein Detection

The presence of relevant bacterial species in a sample also can be quantitatively determined by analysis of one or more proteins unique to the bacteria. Stool sample from a subject is used in the practice of the present invention and can be obtained and processed for analysis according to known methods or as described in the previous section.

### B. Determining the Level of A Bacterial Protein

A protein, e.g., one that is indicative of a bacterium's identity, can be detected using a variety of immunological assays. In some embodiments, a sandwich assay can be performed by capturing the target protein from a test sample with an antibody having specific binding affinity for the protein. The protein then can be detected with a labeled antibody having specific binding affinity for it. Such immunological assays can be carried out using microfluidic devices such as microarray protein chips. A protein of interest (e.g., a protein unique to a bacterial species) can also be detected by gel electrophoresis (such as 2-dimensional gel electrophoresis) and western blot analysis using specific antibodies. Alternatively, standard immunohistochemical techniques can be used to detect a target protein, using the appropriate antibodies. Both monoclonal and polyclonal antibodies (including antibody fragment with desired binding specificity) can be used for specific detection of the target protein. Antibodies and their binding fragments with specific binding affinity to a particular protein can be generated by known techniques.

Other methods may also be employed for measuring the level of a marker protein in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

### V. Monitoring and Treatment of Recurring Colorectal Adenoma

By illustrating the correlation of enrichment of certain bacterial species in human gut and heightened risk of recurrence of colorectal adenoma, the present invention provides a preventive measure for prophylactically treating patients who are at an increased risk of again developing colorectal adenoma, despite prior removal of colorectal cancer or adenoma: the first measure is regular monitoring such as annually scheduled colonoscopy for the patients, such that newly developed colon polyps and cysts can be immediately detected and removed. The second measure is by treating patients with one or more inhibitors to suppress the pertinent bacterial species and reducing their presence/level in the patients' gut.

Inhibitors of the pertinent bacterial species can be of virtually any chemical and structural nature: they may be polypeptides (e.g., antibody, antibody fragment, aptamer), polynucleotides (e.g., antisense DNA/RNA, small inhibitory RNA, or micro RNA), and small molecules. As long as they possess confirmed inhibitory effect against the target bacteria (e.g., suppression of bacterial proliferation or induced death of bacterial cells), such inhibitors may be useful for suppressing development of recurring adenoma in a patient's gut and therefore useful for suppressing or preventing the recurrence of colorectal adenoma.

In addition, upon detecting the enrichment of certain bacterial species in a patient's gut after prior surgical removal of colorectal cancer or polypectomy, which is shown by the present inventors as relevant to the increased likelihood of recurrence of colorectal adenoma, one may identify the patient as having an increased risk of later developing the adenoma again. As a result of this determination, the patient may be subject to subsequent monitoring and therapies or preventive/monitoring measures, such that the recurrence of colorectal adenoma may be prevented, eliminated, ameliorated, reduced in severity and/or frequency, or delayed in the onset. For example, a physician may prescribe both pharmacological and non-pharmacological treatments such as lifestyle modification (e.g., reduce body weight by 5% or more, assume a healthier life style including following a high fibre/low salt diet and maintaining a higher level of physical activities such as walking for at least 150 minutes weekly, and undergo more frequently scheduled screening/examination such as colonoscopy every 1-2 years rather than every 5 years).

### A. Modulators of Pertinent Bacterial Species

Suppression of a bacterial species can be achieved through the use of inhibitor nucleic acids targeting specific bacterial genes such as siRNA, microRNA, miniRNA, lncRNA, antisense oligonucleotides, aptamer. Such nucleic acids can be single-stranded nucleic acids (such as mRNA) or double-stranded nucleic acids (such as DNA) that can translate into an active form of inhibitor of target bacterial RNA under appropriate conditions.

In one embodiment, the inhibitor-encoding nucleic acid is provided in the form of an expression cassette, typically recombinantly produced, having a promoter operably linked to the polynucleotide sequence encoding the inhibitor. In some cases, the promoter is one that directs expression specifically in selected bacterial cells. Administration of such nucleic acids can suppress target bacterial gene expression and therefore suppress the bacterial population. Since virtually all known bacteria have been fully sequenced and information deposited in data banks, one can devise suitable inhibitor nucleic acids based on the sequence information.

Inhibitors of the pertinent bacterial species can be confirmed in assays where a bacterial culture is exposed to a candidate compound, and the compound's effect on the culture is analyzed. For example, an inhibitor can be observed to exhibit an inhibitory or suppressing effect on the bacterial culture, resulting in reduced growth and/or increased bacterial cell death. An inhibitory effect is detected when a negative effect on the bacterial culture is established in the test group. Preferably, the negative effect is at least a 10% decrease; more preferably, the decrease is at least 20%, 50%, 75%, 80% or higher in cell proliferation.

As stated above, these bacterial inhibitors can have diverse chemical and structural features. For instance, an inhibitor can be any small molecule or macromolecule that simply affects the growth or survival of a particular bacterial species. Essentially any chemical compound can be tested as a potential inhibitor. These inhibitors can be identified by screening a combinatorial library containing a large number of potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

### B. Pharmaceutical Compositions

### 1. Formulations

The inhibitors of pertinent bacterial species are useful in the manufacture of a pharmaceutical composition or a medicament. A pharmaceutical composition or medicament can be administered to a subject for the treatment of recurring colorectal adenoma, especially for prophylaxis.

Compounds used in the treatment method of the present invention are useful in the manufacture of a pharmaceutical composition or a medicament comprising an effective amount thereof in conjunction or mixture with excipients or carriers suitable for application.

An exemplary pharmaceutical composition for such therapeutic use comprises (i) an express cassette comprising a polynucleotide sequence encoding an inhibitor (e.g., siRNA, microRNA, miniRNA, lncRNA, antisense oligonucleotides) as described herein, and (ii) a pharmaceutically acceptable excipient or carrier. The terms pharmaceutically-acceptable and physiologically-acceptable are used synonymously herein. The expression cassette may be provided in a therapeutically effective dose for use in a method for treatment as described herein.

An inhibitor can be administered via liposomes, which serve to target the conjugates to a particular tissue, as well as increase the half-life of the composition. Liposomes include emulsions, foams, micelles, insoluble monolayers, liquid crystals, phospholipid dispersions, lamellar layers and the like. In these preparations the inhibitor to be delivered is incorporated as part of a liposome, alone or in conjunction with a molecule which binds to, e.g., a receptor prevalent among the targeted cells, or with other therapeutic or immunogenic compositions. Thus, liposomes filled with a desired inhibitor of the invention can be directed to the site of treatment, e.g., colon, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) Ann. Rev. Biophys. Bioeng. 9: 467, U.S. Pat. Nos. 4,235,871, 4,501,728 and 4,837,028.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E.W. Martin. Compounds and agents of the present invention and their physiologically acceptable salts and solvates can be formulated for administration by any suitable route, including via inhalation, topically, nasally, orally, parenterally, or rectally.

Typical formulations for local or topical administration include creams, ointments, sprays, lotions, and patches. The pharmaceutical composition can, however, be formulated for any type of administration, local as well as systemic, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

### 2. Routes of Administration

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Suitable formulations for transdermal application include an effective amount of a modulator of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., an inhibitor or an activator, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Compounds and agents of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the active ingredient may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The modulators can also be formulated in rectal compositions, for example, suppositories or retention enemas, for example, containing conventional suppository bases, for example, cocoa butter or other glycerides.

Furthermore, the active ingredient can be formulated as a depot preparation. Such long-acting formulations can be administered by implantation (for example, subcutaneously or intramuscularly) or by intramuscular injection. Thus, for example, the active ingredient can be formulated with suitable polymeric or hydrophobic materials (for example as an emulsion in an acceptable oil) or ion exchange resins, or as sparingly soluble derivatives, for example, as a sparingly soluble salt.

In some cases, a pharmaceutical composition or medicament of the present invention comprises (i) an effective amount of a compound as described herein that suppresses the population of one or more of the pertinent bacterial species identified herein, and (ii) another therapeutic agent. When used with a compound of the present invention, such therapeutic agent may be used individually, sequentially, or in combination with one or more other such therapeutic agents (e.g., a first therapeutic agent, a second therapeutic agent, and an anti-bacterial inhibitor of the present invention). Administration may be by the same or different route of administration or together in the same pharmaceutical formulation.

### 3. Dosage

Pharmaceutical compositions or medicaments can be administered to a subject at a therapeutically effective dose to prevent, treat, or control colorectal adenoma recurrence as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular compound in a particular subject. For example, each type of inhibitor or nucleic acid encoding an inhibitor will likely have a unique dosage. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies and repetition rates.

To achieve the desired therapeutic effect, compounds or agents may be administered for multiple days at the therapeutically effective daily dose. Thus, therapeutically effective administration of compounds to treat a pertinent condition or disease described herein in a subject requires periodic (e.g., daily) administration that continues for a period ranging from three days to two weeks or longer. Typically, agents will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

Optimum dosages, toxicity, and therapeutic efficacy of such compounds or agents may vary depending on the relative potency of individual compounds or agents and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any agents used in the methods of the invention, the therapeutically effective dose can be estimated initially from cell culture assays. A dose can be formulated in animal models to achieve a circulating plasma concentration range that includes the IC₅₀ (the concentration of the agent that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of agents is from about 1 ng/kg to 100 mg/kg for a typical subject.

Exemplary dosages for an inhibitor or a nucleic acid encoding an inhibitor described herein are provided. Dosage for an inhibitor-encoding nucleic acid, such as an expression vector, can be between 0.1-0.5 mg with IV administration (e.g., 5-30 mg/kg). Small organic compounds inhibitors can be administered orally at between 5-1000 mg, or by intravenous infusion at between 10-500 mg/ml. Polypeptide inhibitors can be administered by intravenous injection or infusion at 50-500 mg/ml (over 120 minutes); 1-500 mg/kg (over 60 minutes); or 1-100 mg/kg (bolus) five times weekly. Inhibitors can be administered subcutaneously at 10-500 mg; 0.1-500 mg/kg intravenously twice daily, or about 50 mg once weekly, or 25 mg twice weekly.

Pharmaceutical compositions of the present invention can be administered alone or in combination with at least one additional therapeutic compound. Exemplary advantageous therapeutic compounds include systemic and topical anti-inflammatories, pain relievers, anti-histamines, anesthetic compounds, and the like. The additional therapeutic compound can be administered at the same time as, or even in the same composition with, main active ingredient. The additional therapeutic compound can also be administered separately, in a separate composition, or a different dosage form from the main active ingredient. Some doses of the main ingredient can be administered at the same time as the additional therapeutic compound, while others are administered separately, depending on the particular findings of gut bacterial population and characteristics of the individual.

The dosage of a pharmaceutical composition of the invention can be adjusted throughout treatment, depending on various factors including profile of patient's gut bacterial population and physiological response to the therapeutic regimen. Those of skill in the art commonly engage in such adjustments in therapeutic regimen.

### VI. Kits and Devices

The invention provides compositions and kits for practicing the methods described herein to assess the risk of colorectal adenoma recurrence by determining the level or relative abundance of pertinent bacterial species such as one or more of Fn, Bc, Ch, and m3 in a sample, such as a stool sample, obtained from a human patient who have previously had colorectal cancer, cyst, or polyp removed by surgery or polypectomy. For example, the level or relative abundance of one or more of Fn, Bc, Ch, or m3 is determined in a fecal sample taken from a patient, such that the patient may have been treated accordingly, e.g., if the patient is deemed likely to suffer from recurring colorectal adenoma, the patient will be given increased monitor such as more frequently scheduled colonoscopy (such as annually versus once every 5 years), prescribed altered dietary and physical activity regimen, up to and including anti-bacterial treatment to suppress the level of the pertinent bacterial species such as Fn, Bc, Ch, or m3 (for example, administration of an expression cassette such as one contained in a viral vector directing the expression of an inhibitory RNA molecule, especially in colorectal epithelial tissue to inhibit the expression of one or more genes of Fn, Bc, Ch, or m3); otherwise the patient will not be given increased monitoring but rather stay on a regular monitoring schedule such as colonoscopy every 5 years for average risk individuals. In any case, when a patient is screened for possible recurring colorectal adenoma (e.g., by colonoscopy), removal of abnormal colorectal tissue (cysts, polyps, and tumors including adenomas and carcinomas etc.) so discovered can be performed in accordance with established procedures.

In the case of assessing likelihood of colorectal adenoma recurrence among multiple patients who had undergone a procedure to remove colorectal cancer/polyp/cysts, a first patient who has a larger percentage increase in the level or relative abundance of Fn, Bc, Ch, or m3 or their combined score in his post-procedure stool sample compared to his corresponding baseline level or score in his stool sample obtained before the procedure for CRC/polyp/cyst removal is deemed to have a higher chance to suffer from recurring colorectal adenoma than a second patient who has a smaller percentage increase or no increase from the baseline value to the post-procedure value. As such, the first patient is given increased monitoring (e.g., more frequently scheduled colonoscopy, such as once per year rather than once every 5 years) and treatment (e.g., anti-bacterial therapy, especially one targeting one or more of the bacterial species Fn, Bc, Ch, or m3) in contrast to the second patient, who may be given less monitoring and/or treatment, including regularly scheduled colonoscopy and/or no anti-bacterial treatment at all, especially if the second patient is deemed to have no increased risk for recurrence of colorectal adenoma.

Among patients who are determined as having heightened risk for recurrence of colorectal adenoma and are thus given anti-bacterial treatment in order to prevent or reduce the risk of recurrence of colorectal adenoma, their stool samples are optionally further tested after the anti-bacterial treatment such that the level of bacterial species Fn, Bc, Ch, or m3 or their combined score may be ascertained to confirm reduced risk.

In some embodiments, kits for carrying out assays for determining the level or relative abundance of at least one optionally more bacterial species Fn, Bc, Ch, or m3 typically include reagents useful for carrying out an RT-PCR or qPCT for the qualitative and/or quantitative determination of a polynucleotide sequence such as 16S rRNA unique to the bacterial species: for example, at least one oligonucleotide useful for reverse transcription and at least one set of three oligonucleotide primers for PCR to amplify the unique polynucleotide sequence. In some cases, one or more of the oligonucleotides may be labeled with a detectable moiety. In some cases, a hydrolysis probe is included in the kit to allow instant quantitative measure of amplification product. Typically, the hydrolysis probe has a fluorescent label and a quencher. The Examples section of this disclosure provides some examples of such primers and probes.

Typically, the kits also include positive and negative controls for the specific assay method. In addition, the kits of this invention may provide instruction manuals to guide users in analyzing samples and assessing the likelihood of recurrence of colorectal adenoma in a test subject.

In a further aspect, the present invention can also be embodied in a device or a system comprising one or more such devices, which is capable of carrying out all or some of the method steps described herein. For instance, in some cases, the device or system performs the following steps upon receiving a first sample (e.g., a stool sample from a test subject prior to a procedure for removing colorectal cancer/polyp/cyst, such as polypectomy) and a second sample of the same type (e.g., a stool sample from the same test subject after the procedure, for example, about one year, about two years, or about one to about two, three, four, or five years, after the procedure) to assess the subject's likelihood of colorectal adenoma recurrence: (a) determining in the first sample and in the second sample the level or relative abundance of one or more bacterial species Fn, Bc, Ch, or m3 (e.g., based on its unique 16S rRNA sequence) or a combined score of the level or relative abundance of any two, three, or four of these species (e.g., Fn and m3, optionally further including Ch) calculated according to the description provided herein, optionally further including FIT score; (b) comparing the level or combined score from the first sample with the level or combined score from the second sample; and (c) providing an output indicating whether the test subject has a heightened risk for suffering from recurring colorectal adenoma and therefore should be prescribed preventive monitoring and/or treatment as described herein to eliminate or reduce such risk. In some cases, the device or system of the invention performs the task of steps (b) and (c), after step (a) has been performed and the levels or combined scores from step (a) has been entered into the device.

Preferably, the device or system of this invention is partially or fully automated.

### EXAMPLES

The following examples are provided by way of illustration only and not by way of limitation. Those of skill in the art will readily recognize a variety of non-critical parameters that could be changed or modified to yield essentially the same or similar results.

### Example I

### Introduction

Colorectal cancer (CRC) is one of the most common cancers worldwide [1]. Most CRCs begin as adenomas and gradually develop into cancer. Colonoscopy with polypectomy has been used as first-line modality for CRC prevention. Adenomatous polyps can be detected in 20 to 40% of patients undergoing screening colonoscopy, and their occurrence is associated with an increased risk of CRC. Although endoscopic removal of colorectal adenomas significantly reduces the risk of CRC, regular surveillance examinations is needed as risk of recurrence after polypectomy ranges from 37% to 60% [2]. Recently, new biomarkers for CRC diagnosis including stool DNA [3], plasma DNA [4] and fecal bacterial markers [5] have been developed. The U.S. Food and Drug Administration (FDA) has approved two noninvasive tests, a multi-target stool DNA [3] and a plasma DNA test for CRC screening [4]. However, these tests have low diagnostic accuracy for precancerous lesions especially non-advanced adenomas because genetic or epigenetic changes in cancerous cells are rarely present in small pre-cancer lesions.

Altered gut microbiota composition has been implicated in the initiation and progression of adenomas and CRC [6-10]. In particular, a direct causative role of gut microbiota for CRC development was demonstrated in germ-free animal models [11]. Specific bacterial pathogens, such as Fusobacterium nucleatum (Fn) [12-14] and Peptostreptococcus anaerobius [15], have been proposed to promote colorectal tumorigenesis. It was previously reported that fecal bacterial markers were useful as noninvasive tests for adenomas and CRC [5, 7, 16, 17]. Using probe-based duplex quantitative polymerase chain reaction (qPCR) assays for quantification of bacterial marker candidates, a panel of fecal bacterial markers comprising Fn, Lachnoclostridium marker m3, Clostridium Hungatella hathewayi (Ch) and Bacteroides clarus (Bc) (i.e., the so-called 4Bac panel of Fn, m3, Ch and Bc) showed good diagnostic performance for detection of adenoma and CRC [5, 16]. It has been hypothesized that these bacterial markers are also effective in the detection of recurrent adenomas. In this study, the present inventors evaluated the utility of 4Bac panel of Fn, m3, Ch and Bc in predicting risk of adenoma recurrence following colonoscopic polypectomy.

### Materials and Methods

### Study Subjects

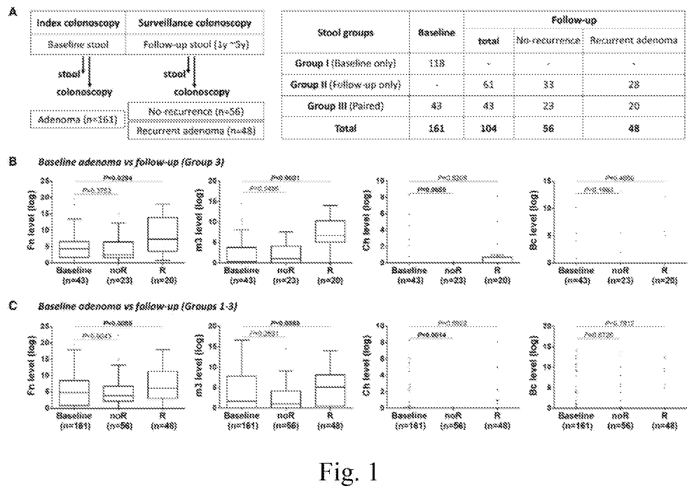

This study included subjects who were part of a polyp surveillance study between 2009 and 2019 conducted at the Prince of Wales Hospital, a secondary and tertiary referral center of The Chinese University of Hong Kong (CREC Ref No: 2010.198). All subjects provided fecal samples before index colonoscopy (baseline). Subjects found to have adenomas on index colonoscopy underwent polypectomy and had regular surveillance colonoscopies according to International polyp surveillance guidelines [2, 18, 19]. At surveillance colonoscopy, no recurrence was defined as colonoscopies where no adenoma, sessile serrated polyps, hyperplastic polyps over 10 mm, or CRC was found. Recurrence was defined as colonoscopies whereby at least one adenoma was found. To identify new lesions, only polypectomies that occurred at least 6 months after the index colonoscopy were included. Colonoscopists had adenoma detection rate of >30%. A stool sample was collected before each surveillance colonoscopy. Included were three groups of subjects—group I: 118 patients with adenomas who had fecal samples collected before index colonoscopy (baseline samples); group II: 61 subjects who had fecal samples collected during surveillance colonoscopy (follow-up samples); and group III: 43 subjects who had paired baseline samples and follow-up samples before colonoscopies) (FIG. 1A). All lesions were confirmed by an experienced pathologist (TKF). Advanced adenomas were defined as adenomas 1 cm or larger in size, with a tubulovillous or villous component, or with high-grade or severe dysplasia. Exclusion criteria included subjects who had taken antibiotics within the past 3 months. Informed consents were obtained from all subjects. The study was approved by the Joint NTEC-CUHK Clinical Research Ethics Committee.

### Human Fecal Sample Collection

Baseline stools (n=161) were collected before index colonoscopy from group I (n=118) and group III (n=43), whereby 104 follow-up stools were collected before surveillance colonoscopy, 2.5±1.6 years after the index colonoscopy, from group II (n=61) and group III (n=43). Forty-eight of the 104 post-polypectomy patients (28 of group II and 20 of group III) were found to have adenomas at follow-up colonoscopies (FIG. 1A), seven of which were advanced adenomas. Detailed clinical characteristics are shown in Table 4. Subjects collected stool samples in standardized containers at home and immediately stored the samples in their home −20° C. freezer. Frozen samples were then delivered to the hospital in insulating polystyrene foam containers and stored at −80° C. immediately until further analysis.

Fecal DNA Extraction and Probe-Based Quantification of Bacterial Markers by Duplex Quantitative PCR (qPCR)

Fecal DNA extraction was performed using the Norgen Stool DNA Isolation Kit (Norgen Biotek Corp, Ontario, Canada) following manufacturer's instruction. DNA quality and quantity were determined using gel electrophoresis and a NanoDrop spectrophotometer. Fecal levels of four bacterial DNA markers (Fn, m3, Bc and Ch) were quantified by qPCR, covering markers previously shown to be enriched in samples from patients with CRC (Fn and Ch), adenoma and CRC (m3) and samples from healthy subjects (Bc). Primer and probe sequences targeting the markers and 16s rDNA internal control have been verified for target specificity in our previous study [5, 16]. Each probe carried a 5′ reporter dye FAM (6-carboxy fluorescein) or VIC (4,7,2′-trichloro-7′-phenyl-6-carboxyfluorescein) and a 3′ quencher dye TAMRA (6-carboxytetramethyl-rhodamine). Primers and hydrolysis probes were synthesized by Invitrogen (Carlsbad, CA). qPCR amplifications were performed on an ABI QuantStudio sequence detection system as previously described, with thermal cycler parameters of 95° C. 10 minutes and (95° C. 15 seconds, 60° C. 1 minute)×45 cycles [5, 16]. Positive controls of the markers and a negative control (H₂O as template) were included within every experiment. Measurements were performed in triplicates for each sample. Relative level of each marker was calculated by using delta Cq method as compared to internal control (Power (2, −(Cq_{target}−Cq_{control})) and shown as Log value of ‘*10e6+1’.

### Scoring Algorithms and Cutoff Values

Combined score of four bacterial markers (4Bac) using a logistic regression model (4Bac score=I₁+β₁*Fn+β₂*m3+β₃*Bc+β₄*Ch) was determined in a previous study [5]. Combined scores of follow-up markers using logistic regression models are as following: I2+β5*m3+β6*Ch, or I3+β7*Fn+β8*m3+β9*Ch (Table 2). Combined score of baseline and follow-up markers using a logistic regression model is as following: I₁₀+(βᵢ*Fn_{followup}−βⱼ*Fn_{Baseline})+(βₖ*m3_{followup}−βₗ*m3_{Baseline})+(βₘ*Ch_{followup}−βₙ*Ch_{Baseline}) (Table 1). In the regression models, I represents the intercepts, R represents the regression coefficients, and markers represent the corresponding Cq values. Cutoff values were determined by receiver operating characteristic (ROC) analyses that maximized the Youden index (J=Sensitivity+Specificity −1) [20].

### Fecal Immunochemical Test (FIT)

The quantitative OC-Sensor tests were performed on an automatic OCsensor instrument (Eiken Chemical, Japan) according to manufacturer's instruction, using a positive cut-off value equivalent to a concentration of 100 ng of haemoglobin per milliliter (ng Hb/mL).

### Statistical Analyses

Values were expressed as median (interquartile range) or mean±SD as appropriate. The differences in bacterial levels were determined by Mann-Whitney U test or paired t-test. Continuous clinical and pathological variables were compared by t-test or one-way ANOVA. ROC curves were used to evaluate the diagnostic values of bacterial markers or models in distinguishing between patients with and without recurrent adenomas. Pairwise comparison of areas under ROC (AUROCs) for each method/marker was performed using a non-parametric approach [21]. All tests were done by Graphpad Prism 5.0 (Graphpad Software Inc., San Diego, CA) or MedCalc Statistical Software V.18.5 (MedCalc Software bvba, Ostend, Belgium; web site: medcalc.org; 2018). P<0.05 was taken as statistical significance.

### Results

Increased Fecal Bacterial Markers in Subjects with Adenoma Recurrence

Levels of four bacterial markers Fn, Ch, m3 and Bc were first compared in baseline stool samples from patients with advanced adenomas and in follow-up stool samples from subjects with and without recurrent adenomas after polypectomy. Compared with baseline stool samples, Fn (P<0.05) and m3 (P<0.0001) were significantly increased in follow-up stools from subjects with recurrent adenomas, but there was no significant change of these markers in follow-up samples of subjects with no recurrence from group III (Mann Whitney tests). The marker Ch was decreased in follow-up samples of subjects with no recurrence (P=0.066), but there were no significant changes in those with adenoma recurrence from group III compared with their baseline stool samples. The marker Bc showed no change in follow-up stool samples regardless of adenoma recurrent status compared with respective baseline samples (FIG. 1B). These findings were further validated in an enlarged dataset involving all samples from groups I to III. Fn and m3 were significantly increased in follow-up stools of subjects with recurrent adenomas, whilst Ch was significantly decreased in follow-up stools of subjects without recurrent adenomas (all P<0.05) compared with baseline stools (FIG. 1C). The marker Bc remained unchanged in follow-up stools compared with baseline stools regardless of adenoma recurrent status. Importantly, fecal levels of Fn, m3 and Ch in subjects with recurrent adenomas showed no difference between those with proximal lesions and those with distal lesions (FIG. 1D1 and FIG. 1D2). These findings indicate that CRC or adenoma-enriched bacterial markers increased in stools of subjects with adenoma recurrence or decreased in stools of subjects without no adenoma recurrence after colonoscopic resection of advanced adenomas.

Fecal Bacterial Marker Levels Differed Between Subjects with and without Adenoma Recurrence

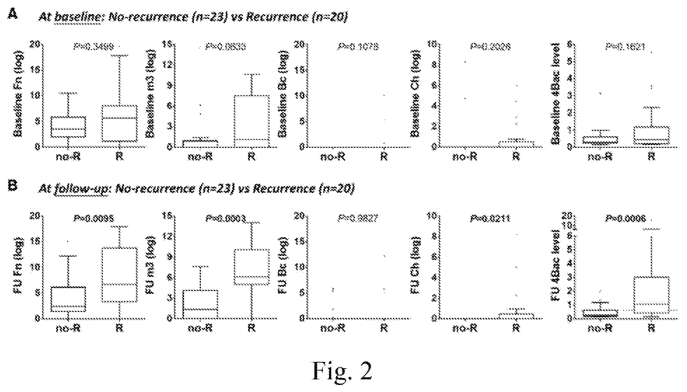

To evaluate if fecal bacterial markers at index colonoscopy and during surveillance colonoscopy correlated with colonoscopic outcomes, subjects with both baseline and follow-up stools were classified into two groups ‘Recurrence’ (n=20; 46.5%) and ‘No-recurrence’ (n=23; 53.5%) according to surveillance colonoscopy and histology results. Based on levels of bacterial markers at baseline stool samples, no significant difference was found in levels of tested bacterial markers between subjects with adenoma recurrence and those without recurrence at follow-up (FIG. 2A). At follow-up time points, it was observed that levels of Fn (P<0.01), m3 (P<0.001) and Ch (P<0.05), as well as the 4Bac combined score (Fn, m3, Ch and Bc) previously devised for CRC/adenoma diagnosis (P<0.001), were significantly higher in subjects with adenoma recurrence than those without recurrence (FIG. 2B). These results indicate that quantification of bacterial markers at surveillance follow-up can help predict patients at high risk of adenoma recurrence.

### Adding FIT Did not Improve Prediction of Adenoma Recurrence

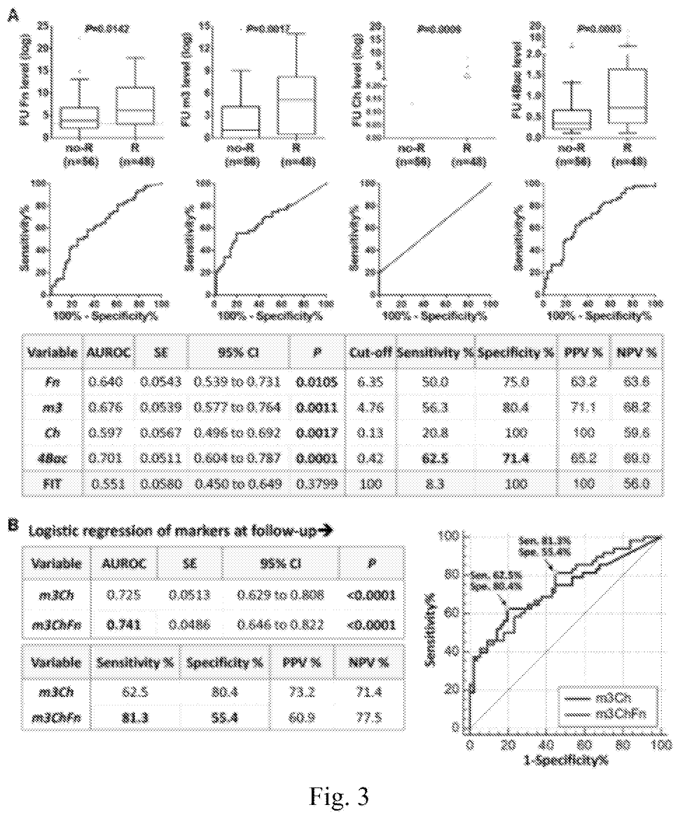

All follow-up stools were further included to assess the performance of bacterial markers at follow-up time points in predicting adenoma recurrence. ROC curve analysis showed that individual Fn, m3, and Ch markers significantly discriminated subjects with adenoma recurrence from those without recurrence with an AUROCs of 0.640, 0.676 and 0.597, respectively (all P<0.05), while FIT failed to discriminate subjects with and without adenoma recurrence (AUROC=0.551, P=0.38) (FIG. 3A). m3 performed better than Fn and Ch in predicting adenoma recurrence, with a sensitivity of 52.0% at a specificity of 80.4%. Although Ch showed a relatively small AUROC, it had a specificity of 100% and sensitivity of 20.8% for predicting adenoma recurrence. In contrast, FIT showed limited sensitivity (8.3%) in detecting recurrent adenomas, most of which were non-advanced adenoma.

At a specificity of 71.4%, the combined score using 4Bac showed good performance in distinguishing between patients with and without recurrent adenomas, with an AUROC of 0.701 (P=0.0001) and a sensitivity of 62.5%. However, as 4Bac did not significantly outperform the other individual markers (P>0.05 by comparison of ROC curves), a different panel of markers or a different scoring algorithm to combine these markers is warranted for better prediction accuracy. Therefore, logistic regression was applied to combine the bacterial markers. Logistic regression model involving m3 and Ch showed an AUROC of 0.725 (P<0.0001), with a sensitivity of 62.5% at specificity of 80.4%. The model combining Fn, m3, and Ch showed the highest AUROC of 0.732 (P<0.0001) in predicting adenoma recurrence, with a sensitivity of 81.3% and specificity of 55.4% (FIG. 3B). Inclusion of FIT result was not associated with improved diagnostic performance of the bacterial markers.

### A Panel of Fecal Bacterial Markers (m3, Fn and Ch) Showed High Accuracy in Predicting Adenoma Recurrence

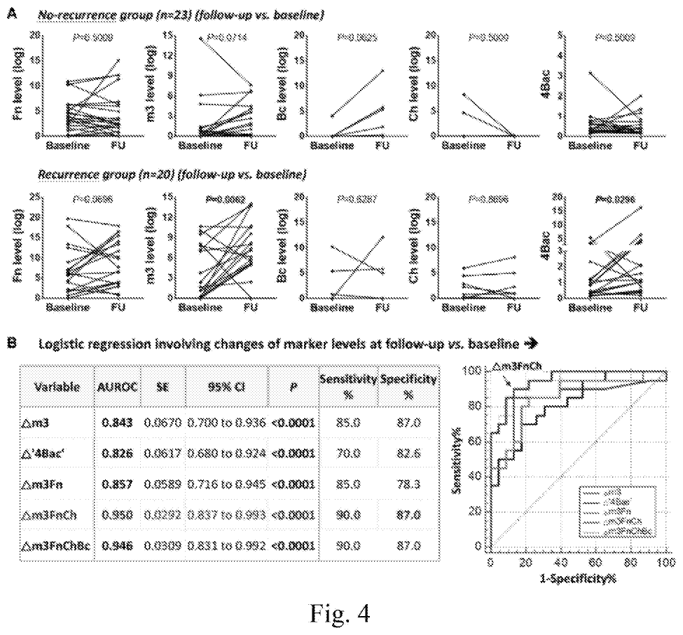

Changes in levels of fecal bacterial markers at surveillance colonoscopy were further compared with fecal samples collected before index colonoscopy. A significant increase was found in level of m3 and combined score 4Bac in the follow-up stool samples compared with baseline samples in subjects who developed recurrence of adenomas confirmed on colonoscopy (P<0.05 by matched-pair tests) (FIG. 4A). In contrast, levels of bacterial markers showed no significant changes in follow-up samples (P>0.05) in subjects who subsequently had a normal colonoscopy during surveillance. Using a logistic regression model that included ‘changes’ in the fecal bacterial markers at follow-up compared with baseline stools, it was found that m3 alone showed a good diagnostic performance for predicting adenoma recurrence, with an AUROC of 0.843 (P<0.0001) and sensitivity of 85.0% at 87.0% specificity. This result outperformed the model utilizing 4Bac score although the figures were not significant (FIG. 4B). Combining Fn or Ch, but not Bc, to m3 in the model further improved the diagnostic performance of m3 alone though this was not significant. The combination of m3, Fn, and Ch performed best in predicting adenoma recurrence, with an AUROC of 0.950 (P<0.0001) and sensitivity of 90.0% and specificity of 87.0% (FIG. 4B).

### Combinations of Fecal Bacterial Markers for Predicting Adenoma Recurrence

Table 1 and Table 2 list the AUROCs, sensitivity and specificity results from various combination of various fecal bacterial markers panel sets and their corresponding method of calculation if more than one biomarker is used. Depending on fecal bacterial markers panel sets used, risk for adenoma recurrence can be predicted by (1) comparing the individual levels or combined scores in samples collected from baseline and follow-up or (2) the individual levels or combined scores in samples collected at follow-up with a standard control (Table 1 or 2).

TABLE 1
Examples of fecal bacterial markers combination and its corresponding calculation method
for prediction of adenoma recurrence (compare between baseline and follow-up)

| Biomarkers | Comparison | Calculation for combined scores | AUROCs | Sensitivity % | Specificity % |
|---|---|---|---|---|---|
| Fn only | Changes | I₄ + (βᵢ*Fn_{followup} − βᵢᵢ*Fn_{Baseline}) | 0.741 | 90.0 | 52.2 |
| m3 only | between | I₅ + (βᵢᵢᵢ*m3_{followup} − βᵢᵥ*m3_{Baseline}) | 0.843 | 85.0 | 87.0 |
| Ch only | baseline & | I₆ + (βᵥ*Ch_{followup} − βᵥᵢ*Ch_{Baseline}) | 0.598 | 25.0 | 100 |
| 4Bac | follow-up | I₇ + (βᵥᵢᵢ*4Bac_{followup} − | 0.826 | 70.0 | 82.6 |
| | | βᵥᵢᵢᵢ*4Bac_{Baseline}) | | | |
| m3 + Fn | | I₈ + (βₐ*Fn_{followup} − β_{b}*Fn_{Baseline}) + | 0.857 | 85.0 | 78.3 |
| | | (β_{c}*m3_{followup} − β_{d}*m3_{Baseline}) | | | |
| m3 + Ch | | I₉ + (βₑ*m3_{followup} − β_{f}*m3_{Baseline}) + | 0.927 | 90.0 | 87.0 |
| | | (β_{g}*Ch_{followup} − βₕ*Ch_{Baseline}) | | | |
| m3 + Fn + Ch | | I₁₀ + (βᵢ*Fn_{followup} − βⱼ*Fn_{Baseline}) + | 0.950 | 90.0 | 87.0 |
| | | (βₖ*m3_{followup} − βₗ*m3_{Baseline}) + | | | |
| | | (βₘ*Ch_{followup} − βₙ*Ch_{Baseline}) | | | |
| m3 + Fn + Ch + Bc | | I₁₁ + (βₒ*Fn_{followup} − βₚ*Fn_{Baseline}) + | 0.946 | 90.0 | 87.0 |
| | | (β_{q}*m3_{followup} − βᵣ*m3_{Baseline}) + | | | |
| | | (βₛ*Ch_{followup} − βₜ*Ch_{Baseline}) − | | | |
| | | (βᵤ*BC_{followup} − βᵥ*Bc_{Baseline}) | | | |

TABLE 2
Examples of fecal bacterial markers combination and its corresponding calculation
method for prediction of adenoma recurrence (compare to standard control)

| Biomarkers | Comparison | Calculation for combined scores | AUROCs | Sensitivity % | Specificity % |
|---|---|---|---|---|---|
| Fn only | Changes of | NA | 0.640 | 50.0 | 75.0 |
| m3 only | fecal | NA | 0.676 | 56.3 | 80.4 |
| Ch only | bacterial | NA | 0.597 | 20.8 | 100 |
| 4Bac | markers | I1 + β1*Fn + β2*m3 + β3*Bc + | 0.701 | 62.5 | 71.4 |
| | level at | β4*Ch | | | |
| m3 + Ch | follow up | I2 + β5*m3 + β6*Ch | 0.725 | 73.2 | 71.4 |
| Fn + m3 + Ch | with a cut- | I3 + β7*Fn + β8*m3 + β9*Ch | 0.741 | 81.3 | 55.4 |
| | off value | | | | |
| | (standard | | | | |
| | control) | | | | |

### Discussion

This is the first study to demonstrate that fecal bacterial markers can effectively predict risk of recurrent adenomas after polypectomy. By quantifying changes in levels of novel bacterial markers during surveillance colonoscopy compared with levels at index colonoscopy, the present inventors found high accuracy of these markers in predicting adenoma recurrence with 90% sensitivity. These findings highlight the role of noninvasive stool markers in improving adenoma surveillance program.

Through metagenome sequencing, microbial markers were previously identified for diagnosis of CRC [7], and a qPCR test was developed for potential clinical application [5, 16]. The qPCR test involved four bacterial markers, including Fn and Ch, found to be enriched in stools of patients with CRC, m3 that was enriched in stools of patients with adenoma and CRC, and Bc that was enriched in stools of normal subjects. The levels of m3 and the combined 4Bac score showed comparable accuracy for detection of advanced lesions and non-advanced lesions suggesting that these bacterial markers were sensitive for detection of small adenomas [5]. Several studies have implicated microbial dysbiosis in the etiology of colorectal adenomas [22]. For instance, changes in the gut microbial community composition have been reported in fecal samples and tissues of adenoma [8, 23]. Unlike existing noninvasive CRC screening tests, such as multi-target stool DNA or plasma DNA tests that target genetic/epigenetic changes from cancerous cells and are rarely present in non-advanced adenomas, the bacterial markers disclosed herein can be used to detect adenomas, and are particularly useful in the detection of early or small precancerous lesions, which account for over 30% of adenomas found on surveillance colonoscopies.

Moderate alteration in gut microbiota was reported at three months after adenoma resection but there was no significant change in the main phyla including Fusobacteria [24]. Although it is expected that gut microbiota composition of patients with adenomas may not change substantially after the lesion was removed, the gut microbiome can be easily reshaped by lifestyle, dietary and nutrient intakes. Altered gut microbiome can promote or inhibit colorectal carcinogenesis, and changes in the microbial community associated with adenomas may represent early events in the pathway leading to CRC. The results of this study demonstrate that CRC or adenoma-enriched bacterial markers, Fn and m3, consistently increased in stools of subjects with adenoma recurrence, whereas Ch decreased in stools of subjects without recurrence. Importantly, fecal levels of m3, Fn and Ch were not different between subjects with recurrent adenomas in the proximal colon compared with those with recurrence in the distal colon, indicating that sensitivity of these markers is not affected by lesion location. These findings support the potential clinical application of bacterial markers Fn, m3 and Ch in the detection of recurrent adenoma. Furthermore, there is potential of modulating the gut microbiome to a healthier state to diminish the risk of developing colorectal neoplasm although this should be evaluated in prospective studies.

The present inventors developed two strategies by including follow-up stools alone or paired baseline and follow-up stools for prediction of adenoma recurrence. Their data show that m3 outperformed Fn and Ch in predicting adenoma recurrence in both strategies, whereas combining Fn and Ch improved the diagnostic performance of m3 in both strategies. The combination of m3, Fn and Ch yielded the best AUROCs compared with other models. The addition of Bc and FIT, however, did not increase the diagnostic sensitivity for recurrent adenoma. It has been demonstrated that Fn induced inflammation and modulated host immune response to promote tumor development [12, 13]. Ch has been shown to promote colonic epithelial cell proliferation in mouse models [25]. It is believed that these bacteria species can trigger host immune responses to further promote the development of recurrent adenomas. Thus, suppressing these bacteria can effectively help reduce the risk of adenoma recurrence.

There is an urgent need for noninvasive biomarkers to monitor for adenoma recurrence. Current guidelines suggest colonoscopic surveillance at variable interval after polyp removal depending on the characteristics of the lesions including size, number, histology, and location [2, 19], but colonoscopy is invasive and regular uptake is low due to suboptimal compliance. A recent national survey demonstrated patient preference for stool-based tests over colonoscopy for CRC screening [26], highlighting the importance of considering patient preference in colorectal screening recommendations.

This study has a number of strengths. It is the first prospective study to follow patients after polypectomy with regular stool sample collection for up to 10 years. Every individual sampled in this study underwent a complete colonoscopy with full visualization of the colon from rectum to cecum, and colonoscopy is regarded as the most robust reference standard for presence or absence of polyps. Polyps removed during colonoscopies were all reviewed and classified by an experienced gastrointestinal pathologist. Finally, this study included predictive algorithms based on bacterial markers known to be enriched/depleted in adenoma and non-adenoma groups.

In summary, this study shows that fecal bacterial markers, including Fn, m3 and Ch, are useful for the diagnosis of recurrent adenomas following polypectomy. This study thus provides the first fecal microbiome-based strategies for surveillance of colorectal neoplasms.

### Example II

### Methods

### Subject Recruitment and Stool Sample Collection for a Prospective Validation Cohort

A prospective study was further conducted to recruit subjects with history of adenoma removal within the past five years and who had arranged regular surveillance colonoscopy at Prince of Wales Hospital, The Chinese University of Hong Kong from May 2021 to October 2021. Consecutive eligible subjects provided follow-up stool samples within one week before bowel preparation of surveillance colonoscopy. Stool samples were excluded if they were collected by subjects who had taken antibiotics within 3 months before stool collection. Colonoscopy and histological examinations were conducted as for the discovery cohort. Subjects collected stool samples in standardized containers with preservative (Norgen, Canada), with which minimal microbial community shifts were observed after 7 days at room temperature as compared to fresh frozen samples in a previous study [5]. Samples were delivered to the hospitals within 24 hours and then stored at −80° C. immediately until further analysis. The study was approved by the Joint NTEC-CUHK Clinical Research Ethics Committee (CREC Ref No: 2021.136). All subjects provided written informed consent.

### Results

Validation of the Recurrence Diagnostic Model with Follow-Up Stools in a Prospective Cohort

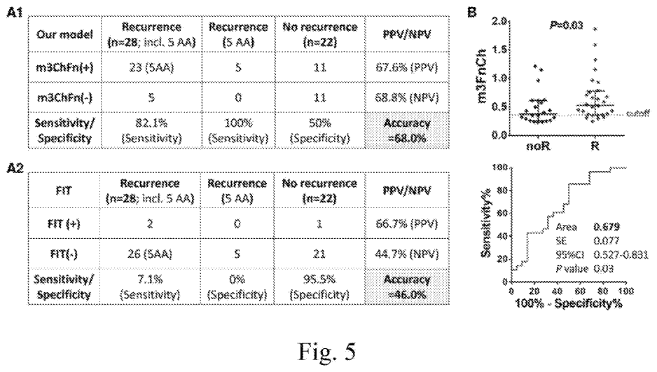

In the validation cohort, 50 consecutive post-polypectomy subjects were recruited and provided qualified stool samples before bowel preparation of surveillance colonoscopy. qPCR test was performed before colonoscopy examination and then compared with colonoscopic diagnostic results. The same logistic regression model involving follow-up levels of m3, Ch and Fn and the same cut-off value from the discovery/training cohort were applied to the validation cohort. Among the 50 subjects, 34 were identified to be of high risk (score higher than cut-off) by the m3ChFn model, 23 of which were confirmed to have adenoma recurrence, resulting in a positive predictive value (PPV) of 67.6% by our model. The other 16 subjects identified to be of low risk by the m3ChFn model, 11 of which were confirmed to have no adenoma recurrence, resulting in a negative predictive value (NPV) of 68.8% by our model. Importantly, 5 patients with recurrent advanced adenoma were all detected with high risk by our model (FIG. 5A1).

The m3ChFn model showed significantly higher combined score for patients with recurrent adenomas as compared to those without recurrence (P=0.03), with an AUROC of 0.679 (95% CI: 0.527-0.831) in this validation cohort (FIG. 5B). The m3ChFn model showed 82.1% sensitivity for recurrent adenoma (100% for recurrent advanced adenoma), with 50% specificity. On the other hand, FIT detected only 2 of the 28 recurrent adenomas (sensitivity=7.1%) and 0 of the 5 recurrent advanced adenoma (sensitivity=0%), though it had a relatively high specificity (95.5%) (FIG. 5A2). The overall diagnostic accuracy was significantly higher by our model (68.0%; 34/50) than by FIT (46.0%; 23/50) (P<0.05 by Fisher's exact test).

All patents, patent applications, and other publications, including GenBank Accession Numbers and equivalents, cited in this application are incorporated by reference in the entirety for all purposes.

TABLE 3
Nucleotide sequences of primers and probes
used in this study

| Primers | sequence (5′→3′) |
|---|---|
| Bc-F | TCCATCCGCAAGCCTTTACT (SEQ ID NO: 1) |
| Bc-R | GCTTCCGGTGCCATTGACTA (SEQ ID NO: 2) |
| m3-F | AATGGGAATGGAGCGGATTC (SEQ ID NO: 3) |
| m3-R | CCTGCACCAGCTTATCGTCAA (SEQ ID NO: 4) |
| Ch-F | GGGCTGCGGAAGCAACTTA (SEQ ID NO: 5) |
| Ch-R | GATGACCTCGCCCTGATCAT (SEQ ID NO: 6) |
| FN-F | TTCAATAAAAGTGGCAGGTCAAG (SEQ ID NO: 7) |
| FN-R | TAACAACACATGCAGGTCAATGG (SEQ ID NO: 8) |
| Ri-F | CGGATTTGCAGTGGCAAGTT (SEQ ID NO: 9) |
| Ri-R | TGATTGCAGACGCCAATGTC (SEQ ID NO: 10) |
| C-F | CGTCAGCTCGTGYCGTGAG (SEQ ID NO: 11) |
| C-R | CGTCRTCCCCRCCTTCC (SEQ ID NO: 12) |
| Probes | sequence (5′→3′) |
| Bc | TTCATCATCACAGCCGACAACGCA |
| | (SEQ ID NO: 13) |
| m3 | AAGCCTGCGGAACCACAGTTACCAGC |
| | (SEQ ID NO: 14) |
| Ch | ACCACCACACAGGACGGAAAGATTCTCC |
| | (SEQ ID NO: 15) |
| FN | ACTCGAACCCCCAACCCTCGGTTT |
| | (SEQ ID NO: 16) |
| Ri | CGTGAAAAATCCGCGCATCTGGC |
| | (SEQ ID NO: 17) |
| C | ttaagtCCCRYAACGAGCGCAACCC |
| | (SEQ ID NO: 18) |

TABLE 4
Clinical characteristics of subjects recruited in this study

| | Group I - Baseline | | Group II - Follow-up | | Group III - Paired | |
|---|---|---|---|---|---|---|
| | samples only (n = 118) | | samples only (n = 61) | | samples (n = 43) | |
| Variables | Baseline | Follow-up | Baseline | Follow-up | Baseline | Follow-up |
| Age | 59.9 ± 5.1 | — | 61.4 ± 6.2 | 64.2 ± 6.0 | 60.0 ± 4.9 | 62.2 ± 4.6 |
| Male gender | 68 | | 35 | 35 | 18 | 18 |
| | (57.6%) | | (57.4%) | (57.4%) | (41.9%) | (41.9%) |
| Recurrent rate | — | — | — | 45.9% | — | 46.5% |
| Adenoma | 118 | — | 61 | 28 | 43 | 20 |
| Non-advanced | 55 | — | 0 | 23 | 0 | 18 |
| | (46.6%) | | (0%) | (82.1%) | (0%) | (90.0%) |
| Advanced | 63 | — | 61 | 5 | 43 | 2 |
| | (53.4%) | | (100%) | (17.9%) | (100%) | (10.0%) |
| Lesion number | 2.4 ± 2.1 | | 1.7 ± 1.0 | 1.9 ± 1.2 | 2.4 ± 1.7 | 1.8 ± 1.1 |
| Lesion size (mm)* | 8.8 ± 8.5 | | 10.2 ± 5.3 | 5.6 ± 4.4 | 13.8 ± 10.6 | 4.4 ± 3.9 |
| Lesion location^{#} | | | | | | |
| Proximal | 32 | — | 12 | 8 | 11 | 7 |
| | (27.1%) | | (21.4%) | (28.6%) | (25.6%) | (35.0%) |
| Distal | 49 | — | 30 | 13 | 17 | 6 |
| | (41.5%) | | (53.6%) | (46.4%) | (39.5%) | (30.0%) |
| both | 37 | — | 14 | 7 | 15 | 7 |
| | (31.4%) | | (25.0%) | (25.0%) | (34.9%) | (35.0%) |

*The size of the largest lesion of each patient is considered.
^{#}Data missing for 5 patients at baseline in Group II.

### REFERENCES

1. Allemani C, Matsuda T, Di Carlo V, et al. Global surveillance of trends in cancer survival 2000-14 (CONCORD-3): analysis of individual records for 37 513 025 patients diagnosed with one of 18 cancers from 322 population-based registries in 71 countries. Lancet 2018; 391:1023-1075.
2. Lieberman D A, Rex D K, Winawer S J, et al. Guidelines for colonoscopy surveillance after screening and polypectomy: a consensus update by the US Multi-Society Task Force on Colorectal Cancer. Gastroenterology 2012; 143:844-857.
3. Imperiale T F, Ransohoff D F, Itzkowitz S H, et al. Multitarget stool DNA testing for colorectal-cancer screening. N Engl J Med 2014; 370:1287-97.
4. deVos T, Tetzner R, Model F, et al. Circulating methylated SEPT9 DNA in plasma is a biomarker for colorectal cancer. Clin Chem 2009; 55:1337-46.
5. Liang J Q, Li T, Nakatsu G, et al. A novel faecal Lachnoclostridium marker for the non-invasive diagnosis of colorectal adenoma and cancer. Gut 2020; 69:1248-1257.
6. Irrazabal T, Belcheva A, Girardin S E, et al. The multifaceted role of the intestinal microbiota in colon cancer. Mol Cell 2014; 54:309-20.
7. Yu J, Feng Q, Wong S H, et al. Metagenomic analysis of faecal microbiome as a tool towards targeted non-invasive biomarkers for colorectal cancer. Gut 2017; 66:70-78.
8. Nakatsu G, Li X, Zhou H, et al. Gut mucosal microbiome across stages of colorectal carcinogenesis. Nat Commun 2015; 6:8727.
9. Dai Z, Coker 00, Nakatsu G, et al. Multi-cohort analysis of colorectal cancer metagenome identified altered bacteria across populations and universal bacterial markers. Microbiome 2018; 6:70.
10. Tilg H, Adolph T E, Gerner R R, et al. The Intestinal Microbiota in Colorectal Cancer. Cancer Cell 2018; 33:954-964.
11. Wong S H, Zhao L, Zhang X, et al. Gavage of Fecal Samples From Patients With Colorectal Cancer Promotes Intestinal Carcinogenesis in Germ-Free and Conventional Mice. Gastroenterology 2017; 153:1621-1633 e6.
12. Kostic A D, Chun E, Robertson L, et al. Fusobacterium nucleatum potentiates intestinal tumorigenesis and modulates the tumor-immune microenvironment. Cell Host Microbe 2013; 14:207-15.
13. Rubinstein M R, Wang X, Liu W, et al. Fusobacterium nucleatum promotes colorectal carcinogenesis by modulating E-cadherin/beta-catenin signaling via its FadA adhesin. Cell Host Microbe 2013; 14:195-206.
14. Yu T, Guo F, Yu Y, et al. Fusobacterium nucleatum Promotes Chemoresistance to Colorectal Cancer by Modulating Autophagy. Cell 2017; 170:548-563 e16.
15. Tsoi H, Chu E S H, Zhang X, et al. Peptostreptococcus anaerobius Induces Intracellular Cholesterol Biosynthesis in Colon Cells to Induce Proliferation and Causes Dysplasia in Mice. Gastroenterology 2017; 152:1419-1433 e5.
16. Liang Q, Chiu J, Chen Y, et al. Fecal Bacteria Act as Novel Biomarkers for Noninvasive Diagnosis of Colorectal Cancer. Clin Cancer Res 2017; 23:2061-2070.
17. Liang J Q, Wong S H, Szeto C H, et al. Fecal microbial DNA markers serve for screening colorectal neoplasm in asymptomatic subjects. J Gastroenterol Hepatol 2020.
18. Rutter M D, East J, Rees C J, et al. British Society of Gastroenterology/Association of Coloproctology of Great Britain and Ireland/Public Health England post-polypectomy and post-colorectal cancer resection surveillance guidelines. Gut 2020; 69:201-223.
19. Gupta S, Lieberman D, Anderson J C, et al. Recommendations for Follow-Up After Colonoscopy and Polypectomy: A Consensus Update by the US Multi-Society Task Force on Colorectal Cancer. Gastrointest Endosc 2020; 91:463-485 e5.
20. Youden W J. Index for rating diagnostic tests. Cancer 1950; 3:32-5.
21. DeLong E R, DeLong D M, Clarke-Pearson D L. Comparing the areas under two or more correlated receiver operating characteristic curves: a nonparametric approach. Biometrics 1988; 44:837-45.
22. Dulal S, Keku T O. Gut microbiome and colorectal adenomas. Cancer J 2014; 20:225-31.
23. Shen X J, Rawls J F, Randall T, et al. Molecular characterization of mucosal adherent bacteria and associations with colorectal adenomas. Gut Microbes 2010; 1:138-47.
24. Yu S Y, Xie Y H, Qiu Y W, et al. Moderate alteration to gut microbiota brought by colorectal adenoma resection. J Gastroenterol Hepatol 2019; 34:1758-1765.
25. Xia X, Wu W K K, Wong S H, et al. Bacteria pathogens drive host colonic epithelial cell promoter hypermethylation of tumor suppressor genes in colorectal cancer. Microbiome 2020; 8:108.
26. Zhu X, Parks P D, Weiser E, et al. National Survey of Patient Factors Associated with Colorectal Cancer Screening Preferences. Cancer Prev Res (Phila) 2021.

1. A method for assessing risk for colorectal adenoma recurrence in an individual after removal of colorectal cancer or adenoma, comprising the steps of:
(a) obtaining in a first stool sample taken from the individual prior to the removal of colorectal cancer or adenoma a baseline level of one or more of four bacterial species of a Lachnoclostridium species carrying genetic marker m3 (m3), Fusobacterium nucleatum (Fn), Clostridium hathewayi (Ch), and Bacteroides clarus (Bc);
(b) obtaining in a second stool sample taken from the individual after the removal of colorectal cancer or adenoma a follow-up level of the one or more of the four bacterial species;
(c) calculating a combined score from the baseline level and follow-up level of any one of more of the four bacterial species Fn, m3, Bc, and Ch; and
(d) detecting the value to be higher than a standard control value and determining the individual as having increased risk for colorectal adenoma recurrence.

2. The method of claim 1, wherein the combined score of the baseline and follow-up levels of any one, two, three, or four of the four bacterial species is calculated by a method listed in Table 1.

3. The method of claim 1, wherein the genome of m3 comprises the nucleotide sequence of SEQ ID NO:19, or wherein the genome of Ch comprises the nucleotide sequence of SEQ ID NO:20, or wherein the genome of Fn comprises the nucleotide sequence of SEQ ID NO:21, or wherein the genome of Bc comprises the nucleotide sequence of SEQ ID NO:22.

4. The method of claim 1, wherein the individual had colorectal adenoma removed by polypectomy.

5. The method of claim 1, wherein steps (a) and (b) each comprises obtaining the level of a DNA, RNA, or protein unique to at least one of the bacterial species Fn, m3, Bc, and Ch.

6. The method of claim 1, wherein steps (a) and (b) each comprises a polymerase chain reaction (PCR) for measuring the level or levels of the bacterial species.

7. The method of claim 6, wherein the PCR is a quantitative polymerase chain reaction (qPCR) or reverse transcription polymerase chain reaction (RT-PCR).

8. The method of claim 1, wherein the second stool sample is taken from the individual about one to five years after the removal of the colorectal cancer or adenoma.

9. The method of claim 8, wherein the second stool sample is taken from the individual about one year after the removal of the colorectal cancer or adenoma.

10. A method for assessing risk for colorectal adenoma recurrence in an individual after removal of colorectal cancer or adenoma, comprising the steps of:
(a) obtaining in a stool sample taken from the individual after the removal of colorectal cancer or adenoma a value of (1) level of one or more of three bacterial species of a Lachnoclostridium species carrying genetic marker m3 (m3), Fusobacterium nucleatum (Fn), and Clostridium hathewayi (Ch); or (2) combined score of levels of two bacterial species m3 and Ch, which is calculated by I₂+β₅*m3+β₆*Ch; or (3) combined score of levels of three bacterial species Fn, m3, and Ch, which is calculated by I₃+β₇*Fn+β₈*m3+β₉*Ch; or (4) combined score of levels of four bacterial species Fn, m3, Bc, and Ch, which is calculated by I₁+β₁*Fn+β₂*m3+β₃*Bc+β₄*Ch; and
(b) detecting the value to be higher than a standard control value and determining the individual as having increased risk for colorectal adenoma recurrence.

11. The method of claim 10, wherein the genome of m3 comprises the nucleotide sequence of SEQ ID NO:19, or wherein the genome of Ch comprises the nucleotide sequence of SEQ ID NO:20, or wherein the genome of Fn comprises the nucleotide sequence of SEQ ID NO:21, or wherein the genome of Bc comprises the nucleotide sequence of SEQ ID NO:22.

12. The method of claim 10, wherein the individual had colorectal adenoma removed by polypectomy.

13. The method of claim 10, wherein step (a) comprises obtaining the level of a DNA, RNA, or protein unique to at least one of the bacterial species Fn, m3, Bc, and Ch.

14. The method of claim 10, wherein step (a) comprises a polymerase chain reaction (PCR) for measuring the level or levels of the bacterial species.

15. The method of claim 14, wherein the PCR is a quantitative polymerase chain reaction (qPCR) or reverse transcription polymerase chain reaction (RT-PCR).

16. The method of claim 10, wherein the stool sample is taken from the individual about one to five years after the removal of the colorectal cancer or adenoma.

17. The method of claim 10, wherein the stool sample is taken from the individual about one year after the removal of the colorectal cancer or adenoma.

18-20. (canceled)

21. A kit for assessing risk for colorectal adenoma recurrence in an individual after removal of colorectal cancer or adenoma, comprising (1) a first container containing a reagent for measuring level of the bacterial species Fn; and (2) a second container containing a reagent for measuring level of the bacterial species m3.

22-26. (canceled)

27. A method for reducing risk of colorectal adenoma recurrence in an individual after removal of colorectal cancer or adenoma, comprising administering to the individual an effective amount of an inhibitor that suppresses or eliminates one or more of the bacterial species Fn, m3, and Ch in the individual.

28-32. (canceled)

33. A kit for reducing risk of colorectal adenoma recurrence, comprising (1) a first container containing one or more reagents for measuring level of the one or more of the bacterial species Fn, m3, and Ch; and (2) a second container containing a composition comprising an effective amount of an inhibitor that suppresses or eliminates one or more of the bacterial species Fn, m3, and Ch.

34-35. (canceled)
